(19) United States
(12) Patent Application Publication
(10) Pub. No.: US 2021/0386452 A1
(43) Pub. Date: Dec. 16, 2021

(54) A HAND-HELD, DIRECTIONAL, MULTI- FREQUENCY PROBE FOR SPINAL NEEDLE PLACEMENT

(71) Applicant: THE REGENTS OF THE UNIVERSITY OF MICHIGAN, Ann Arbor, MI (US)

(72) Inventors: Robert Dick, Chelsea, MI (US); Michael Burns, Novi, MI (US); Leif Saager, Ann Arbor, MI (US); Benjamin Simpson, Ann Arbor, MI (US)

(21) Appl. No.: 17/279,642

(22) PCT Filed: Oct. 9, 2019
(86) PCT No.: PCT/US19/55292

§ 371 Date: Mar. 25, 2021

Related U.S. Application Data
(60) Provisional application No. 62/745,143, filed on Oct. 12, 2018.

(51) Int. Cl. A61B 17/34; A61M 25/06

(52) U.S. Cl. CPC A61B 17/3403; A61M 25/065; A61B 17/3401; A61M 2205/332; A61M 2205/3327; A61B 2017/00022; A61M 2205/50; A61M 2205/587; A61M 2205/18; A61M 2205/3584; A61M 2210/02

(57) ABSTRACT

A predictive needle insertion device including a needle having a proximal needle end and a distal needle end is disclosed. A probe is movably coupled to the needle such that the probe is capable of extending beyond the distal needle end. An actuator is operable to actuate the probe to apply a mechanical force to a tissue composition. A force sensor and position sensor are configured to determine a resistive force of the tissue composition and an insertion distance of the probe, respectively. A processor is communicatively coupled to the force sensor and the position sensor, and is configured to receive sensor data indicative of a mechanical response to the mechanical force and the insertion distance of the probe, and to implement a predictive model that, based on the sensor data, predicts a forward distance to a remote position of a remote tissue portion of the tissue composition.

### REFERENCE TO RELATED APPLICATIONS

This application claims the benefit of U.S. Provisional Application No. 62/745,143, filed Oct. 12, 2018. The entirety of the foregoing provisional application is incorporated by reference herein.

### FIELD OF THE DISCLOSURE

The present disclosure generally relates medical devices, and, more particularly, to needle insertion devices, including predictive needle insertion devices, and further including machine-learning based needle insertion devices.

### BACKGROUND

A number of medical procedures involve gaining access into and around a patient's spinal canal. Accurate and reliable determination of entry or positioning of a medical instrument in the spinal canal or the epidural space is crucial for optimal delivery of care.

For instance, delivery of epidural anesthesia, a type of anesthesia commonly used in childbirth, involves the insertion of a catheter into the epidural space. To introduce the catheter, a special epidural needle is advanced through the back and into the epidural space; the catheter is then inserted through the needle and into the epidural space. During its passage into the body, the needle passes through skin and soft tissue before entering tough ligament. The epidural space is at variable lengths typically just beyond the ligament. The needle must be advanced far enough to reach the epidural space, while advancing too distally should be avoided. If the needle is advanced too far, it will pass through the epidural space and puncture a thin layer of tissue (i.e., the dura mater, or “dura”), entering the subarachnoid space, and causing a cerebrospinal fluid (CSF) leak.

Accurate positioning of a catheter in the epidural space is a process requiring precision. Most doctors identify the epidural space using a “loss of resistance” technique, in which the epidural needle is attached to a “loss of resistance” syringe typically filled with air, water, or saline and having a plunger that moves back and forth with very little resistance. The needle and syringe are slowly advanced into the patient's back while the plunger is occasionally depressed to test for a “loss-of-resistance.” If the needle is in the soft tissue or the tough ligament located between the skin and the epidural space, the plunger will not depress easily. If the needle is in the epidural space, however, the plunger will depress more easily. Once the needle is in the epidural space, an epidural catheter is inserted through the needle and into the epidural space. The catheter is then used to deliver anesthesia or other drugs. Sometimes the drug is injected directly into the epidural space through a needle and a catheter is not inserted.

Unfortunately, complications due to faulty positioning or placement of the catheter are not uncommon during epidural procedures. One of the most frequent complications occurs when the epidural needle is accidentally inserted past the epidural space and through the dura, resulting in a puncture in the spinal canal and subsequent cerebrospinal fluid (CSF) leak. Following accidental dural puncture, patients have a greater than 50% chance of developing a post-dural puncture headache (PDPH) resulting from CSF loss. These headaches are often severe and associated with nausea and vomiting, vision and hearing changes, low back pain, dizziness, and cranial nerve palsies. Most of these headaches subside in about a week, but in some instances can last for months or years. Additionally, if left untreated, the headaches can predispose patients to subdural hematoma and possibly death.

Another common error during epidural anesthesia occurs when a catheter is introduced in an area other than the epidural space, like the surrounding muscles. This error happens because, due to tissue structure differences, these areas can give a false “loss of resistance” upon epidural needle entry. Unfortunately, it is difficult and time consuming to identify misplaced catheters. The current most reliable practice for verifying that a catheter is correctly placed in the epidural space is an injection of local anesthetic and subsequent verification of drug effect. The drug will not take effect if the catheter is not in the epidural space, and since peak effect of correctly delivered drug can take up to 20 minutes, verification by this method can be time consuming. Such a delay can be impractical for a patient in severe pain, and may in fact be dangerous for a woman in need of an urgent caesarean section. In addition to prolonging pain relief, such misplacement necessitates additional procedures, such as additional attempt at epidural anesthesia or even emergency general anesthesia. In addition, such misplacement can result in intravascular injection that can lead to devastating complications such as seizure and local anesthetic toxicity. Further, such misplacement could further add risks of hematoma, infection, and/or reversible or permanent nerve damage.

Both problems, puncturing the dura and putting the catheter in the wrong place, may result because the “loss-of-resistance” technique is simply not particularly sensitive. Further, there is a lack of a suitable alternative that does not involve impractical complexity. For example, ultrasound is sometimes used during epidural needle placement. The utility of such ultrasound procedures, however, is low because it generally requires extensive training and additional personnel; it also dramatically changes the procedure workflow, requiring real-time manual interpretation of complex data and equipment that is generally bulky and costs tens of thousands of dollars.

Other approaches may use optical coherence tomography. However, such approaches are limited because they provide less than 0.5 mm of forewarning before contact with, and/or puncture of, sensitive tissue (e.g., the dura), which is generally inadequate for needle steering purposes.

For the foregoing reasons, there is a need for an improved needle insertion device.

### SUMMARY

Accordingly, improved devices, and related methods, are provided herein for facilitation of access and/or positioning of such devices in a spinal canal of a patient. For example, the needle insertion devices disclosed in various embodiments herein, reduce failed needle-placement attempts experienced during spinal medical procedures, which in turn reduces labor costs and risk of potential complications.

Most epidurals today are performed without assistive technology, meaning that doctors, or other medical personnel, must rely on only tactile feedback and their knowledge of spinal anatomy while inserting a needle, which can be detrimental to current medical practice. The needle insertion devices described herein, however, may significantly improve efficiency and reduce complications associated with medical procedures, such as epidural access procedures, lumbar punctures, or other such similar procedures. In various aspects, the needle insertion devices described herein are configured to provide machine-learning based predictions and classifications, including predictions and classifications associated with needle and/or probe positioning within several millimeters (e.g., 2 to 7 mm) of sensitive tissue (e.g., bone). As a result, such needle insertion devices, including machine-learning based needle insertion devices, as described herein, are operable to forewarn and/or alert medical personnel of the upcoming sensitive tissue to avoid complications and dangers associated with spinal medical procedures.

As described herein, a needle insertion device may include a needle having a proximal needle end and a distal needle end. The needle insertion device may further include a probe movably coupled to the needle such that the probe is capable of extending beyond the distal needle end.

The needle insertion device may further include an actuator operable to actuate the probe to apply a mechanical force to a tissue composition. In various embodiments, the tissue composition includes a local tissue portion (e.g., soft tissue) and a remote tissue portion (e.g., bone), the local tissue portion being situated at a local position to the distal needle end and the remote tissue portion being situated at a remote position to the distal needle end.

The needle insertion device may further include a force sensor associated with the probe. The force sensor may be configured to detect a mechanical response to the mechanical force indicative of resistive force.

The needle insertion device may further include a position sensor associated with the probe. In various embodiments, the position sensor is configured to measure an insertion distance of the probe beyond the distal needle end.

The needle insertion device may further include a processor communicatively coupled to the force sensor and the position sensor. The processor may be configured to receive sensor data indicative of the mechanical response to the mechanical force and the insertion distance of the probe. In still further embodiments, the processor may be configured to implement a machine-learning model that, based on the sensor data, predicts a forward distance to the remote position of the remote tissue portion (e.g., bone).

The needle insertion device(s), as disclosed herein, provide several benefits to medical practitioners (e.g., anesthesiologists, doctors, nurses, etc.). For example, the needle insertion device(s) may be used in various medical procedures requiring injecting medications into specific locations of a patient (e.g., near or around a patient's spine) with minimal damage, including, for example, during epidural anesthesia. As a further example, in some embodiments, the needle insertion device(s) are capable of determining a forward distance to bone during needle insertion, with the goal of allowing medical practitioners to steer needles to appropriate locations.

In some embodiments, the needle insertion device(s) are operable to provide medical practitioners with feedback about the composition of remote tissue in front of a needle during insertion. For example, as described herein, needle insertion device(s) are operable to measure a mechanical response of tissue during needle insertion, and, thereby provide feedback to medical practitioners during a medical procedure. Such information or data aids medical practitioners in safe and efficient needle insertion.

As described herein, in particular embodiments, a medical practitioner may use the needle insertion device to perform a medical procedure requiring spinal canal access. The actuator of the needle insertion device may apply a mechanical force (e.g., which in some embodiments may include multi-frequency force) to a patient's tissue beyond the needle using a probe, which can be, in some embodiments, be a blunt, inter-needle probe. An attached force sensor may record the resistive force of the tissue while a position sensor may measure the probe's insertion distance beyond the needle. The sensor data from both sensors may be collected and analyzed by a local or remote processor of the needle insertion device to determine tissue composition and related distances thereof. For example, in various embodiments, the needle insertion devices can sense or detect tissue several millimeters distant from an associated needle. In such embodiments, the needle insertion devices do not rely on contact with the tissue of interest (e.g., bone) in order to sense or detect such tissue.

In some embodiments, by periodically and automatically probing the tissue as the needle is inserted, the needle insertion device is able to provide continuous feedback on upcoming tissue (e.g., bone). In such embodiments, the sensor data is fed into a machine-learning algorithm that determines how far the needle is from bone thus giving the medical practitioner valuable feedback.

In various embodiments, the needle insertion device is configured as a handheld device. In such handheld embodiments, the needle insertion device may be configured so as to be compatible with existing needle insertion procedures or workflows (e.g., an anesthesiologist's general procedure and/or workflow).

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1A illustrates an example needle insertion device, such as a predictive needle insert device or a machine-learning based needle insertion device, in accordance with various embodiments disclosed herein.

FIG. 1B illustrates an example tissue composition as associated with the needle insertion device of FIG. 1A in accordance with various embodiments disclosed herein.

FIG. 2A illustrates a first embodiment of a display associated with the example needle insertion device of FIG. 1A in accordance with various embodiments disclosed herein.

FIG. 2B illustrates a second embodiment of a display associated with the example needle insertion device of FIG. 1A in accordance with various embodiments disclosed herein.

FIG. 2C illustrates a third embodiment of a display associated with the example needle insertion device of FIG. 1A in accordance with various embodiments disclosed herein.

FIG. 2D illustrates a fourth embodiment of a display associated with the example needle insertion device of FIG. 1A in accordance with various embodiments disclosed herein.

FIG. 2E illustrates a fifth embodiment of a display associated with the example needle insertion device of FIG. 1A in accordance with various embodiments disclosed herein.

FIG. 3A illustrates an example display of sensor data indicative of insertion distance of the probe of the example needle insertion device of FIG. 1A in accordance with various embodiments disclosed herein.

FIG. 3B illustrates an example display of sensor data indicative of a mechanical response to a mechanical force applied by the probe of the example needle insertion device of FIG. 1A in accordance with various embodiments disclosed herein.

FIG. 4 illustrates an example display of machine-learning based predictions and classifications regarding a tissue composition as associated with the example needle insertion device of FIG. 1A in accordance with various embodiments disclosed herein.

FIG. 5 illustrates an example display showing error values for different machine-learning based classifications as associated with the example needle insertion device of FIG. 1A in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION

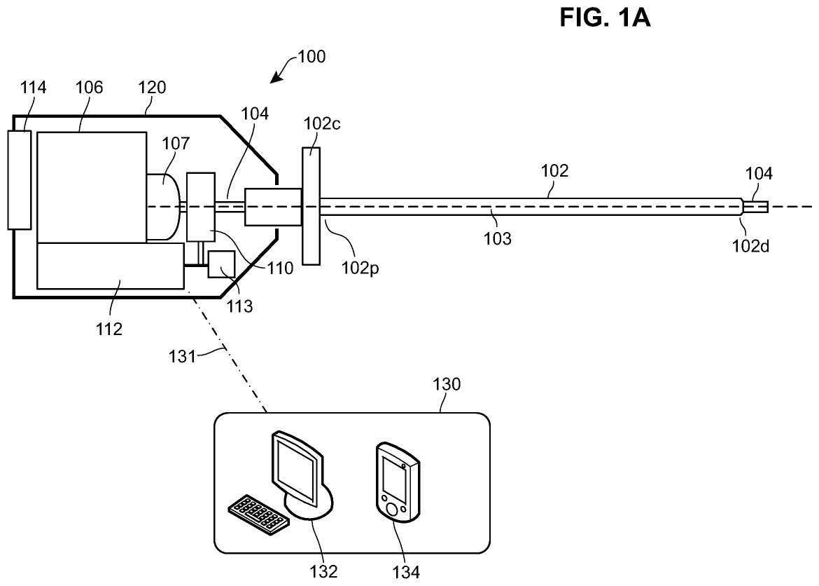

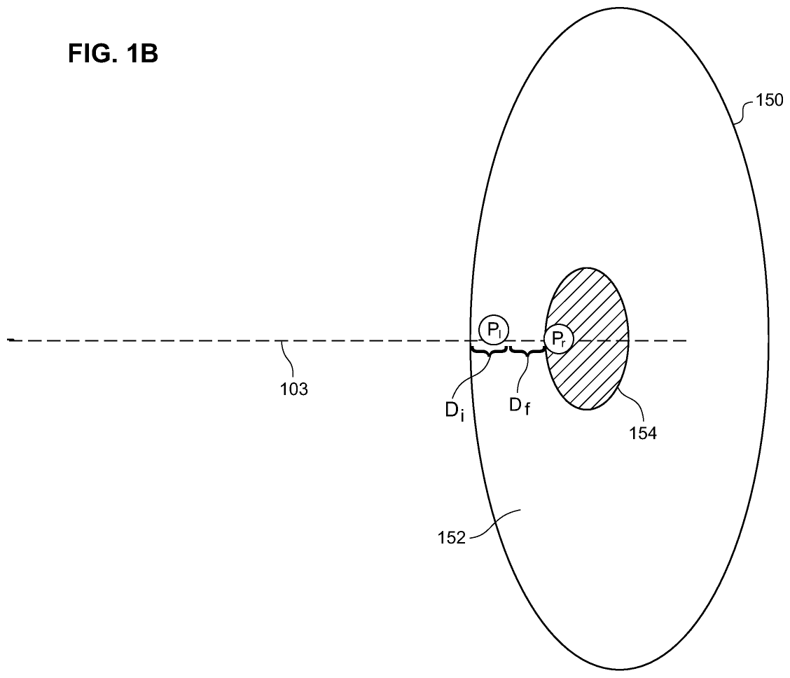

FIG. 1A illustrates an example needle insertion device 100, such as a predictive needle insertion device or a machine-learning needle insertion device, in accordance with various embodiments disclosed herein. In various embodiments, the needle insertion device is a predictive needle insertion device. In various embodiments, the needle insertion device is a predictive needle insurance device, which may be or comprise a machine-learning based needle insertion device. The needle insertion device of various embodiments may include a needle 102 having a proximal needle end 102p and a distal needle end 102d. In some embodiments, the needle may be a 17 gauge needle. Additionally, or alternatively, needle 102 may be other sizes, gauges, etc. For example, needle 102 can be any type of medical needle, such as a standard Tuohy needle commonly used in epidural procedures. In some embodiments, the needle may be configured to receive a catheter through proximal needle end 102p and distal needle end 102d for the delivery of anesthesia or other drugs. While some example embodiments provided herein of needle insertion device 100 are directed to needle insertion into the epidural space, it will be appreciated by those in the art that needle insertion device 100 is also suitable and may be used for insertion of needle 102 into any target tissue of interest, for example, for the administration of a pharmaceutical or imaging agent to the target tissue or for withdrawal (e.g., biopsy) of the target tissue.

The needle insertion device may further include a probe 104 movably coupled to needle 102 such that probe 104 is capable of extending beyond distal needle end 102d. In some embodiments, the probe 104 is movable between a first position proximal to the distal needle end 102d and a second position distal to the distal needle end 102d. In some embodiments, probe 104 may further be capable of retracting into distal needle end 102d. In certain embodiments, probe 104 may be configured as an inter-needle probe that is operable to extend through proximal needle end 102p and distal needle end 102d. For example, in certain embodiments, probe 104 may consist of a thin rod that may be threaded through needle 102.

Needle 102 and probe 104 may be configured to allow for probe 104 to be moved in relation to the needle 102 without undue friction. In such embodiments, needle 102 may be a straight needle and/or probe 104 may be a probe with sufficient lateral flexibility, each such configuration allowing for reduced friction between needle 102 and probe 104. For example, in a particular embodiment, needle 102 may be a straight needle and probe 104 may be a blunt titanium probe. In another particular embodiment, needle 102 may be a Tuohy needle or other needle having a curved tip, and probe 104 may have sufficient lateral flexibility or deformability to smoothly advance through needle 102.

In further embodiments, needle 102 and probe 104 may be removable from the needle insertion device 100, thus allowing for catheter insertion or the use of disposable needles and probes. For example, in some embodiments, needle 102 may be a disposable needle and/or probe 104 may be a disposable probe. In such embodiments, each of needle 102 and probe 104 may be configured as removable from needle insertion device 100. For example, in the embodiment of FIG. 1A, needle 102 may be removable from needle coupling 102c and probe 104 may be removable from actuator coupling 107, as described further herein. In is to be understood that needle 102 and probe 104 may be coupled or connected at additional or alternative locations with respect to needle insertion device 100, so as to be removable, and, therefore disposable in accordance with the disclosures herein.

The needle insertion device 100 may further include an actuator 106 operable to actuate probe 104 to apply a mechanical force to a tissue composition (e.g., tissue composition 150 of FIG. 1B). In various embodiments, actuator 106 may extend, and possibly retract, probe 104 through, or along a same or similar axis (e.g., axis 103) as, needle 102. In some embodiments, actuator 106 is a solenoid based actuator. In other embodiments, other types of actuators, such as linear motors, spring-based energy storage, and pneumatic/hydraulic pistons, may be used. In some embodiments, other sound, infra-sound, or ultra-sound mechanical transducers are used.

Actuator 106 may or may not include an actuator coupling 107. In the embodiment of FIG. 1, actuator coupling 107 mechanically connects actuator 106 to probe 104 such that actuator 106 is coupled directly to probe 104. In other embodiments, however, actuator 106 may be coupled indirectly to probe 104 (not shown).

FIG. 1B illustrates an example tissue composition 150 as associated with the needle insertion device 100 of FIG. 1A in accordance with various embodiments disclosed herein. Tissue composition 150 includes a local tissue portion 152 and a remote tissue portion 154. Tissue composition 150 may represent a top down, cross section view of a patient's torso and spine. Local tissue portion 152 may represent soft tissue (e.g., epidermis, dermis, muscle, etc.) and remote tissue portion 154 may represent bone (e.g., a spine vertebrae). As illustrated in FIGS. 1A and 1B, during a medical procedure utilizing the needle insertion device 100, local tissue portion 152 is situated generally at a local position Pi to the distal needle end 102d of the needle insertion device 100, and remote tissue portion 154 is situated generally at a remote position Pᵣ to distal needle end 102d of the needle insertion device 100. In the embodiment of FIGS. 1A and 1B, each of local position Pi and remote position Pᵣ are situated along axis 103 which extends along needle 102. Additionally, or alternatively, it is to be understood that local position Pi and remote position Pᵣ are respective positions relative to the placement of needle 102, and in particular, relative to the placement distal needle end 102d, with respect to tissue composition 150. It is to be understood, therefore, that local position Pi and remote position Pᵣ change accordingly with the positioning of the needle insertion device 100, and the position of needle 102 attached thereto.

With reference to FIG. 1A, the needle insertion device 100 may further include a force sensor 110 associated with probe 104. The force sensor 110 may be configured to detect a resistive force of tissue composition 150. In various embodiments, the resistive force, as experienced by force sensor 110, may be measured as a mechanical response to the mechanical force applied by probe 104. For example, in some embodiments, the mechanical response may include a responsive force, as provided as a physical reaction/force from the tissue composition 150 in response to contact or proximity with the probe 104 and/or via actuation of the probe 104 as described herein. In particular, force sensor 110 may measure the force response (e.g., mechanical response) of the tissue, where such force response is caused by tissue stress, strain, or both stress and strain, resulting from actuation of probe 104. In this way, probe 104 acts as a stimulant to the tissue composition 150 to encourage the mechanical response which is read by force sensor 110.

In some embodiments, tissue stress may be measured with force sensor 110 in axial alignment (e.g., along axis 103) with probe 104 to measure tissue stress and/or strain using a magnetic linear encoder. The encoder may be part of, or external to, force sensor 110. In some embodiments, the resistive force may be measured as a viscoelastic response as further described herein.

In addition, or in the alternative, other types of force transducers may be employed to measure tissue stress and/or strain for the purposes of determining resistive forces. Such force transducers may include, for example, accelerometers, string potentiometers, or current/voltage sense resistors for measuring real-time actuator power consumption (which can then be converted into stress and/or strain). Additionally, or alternatively, different sensors may further be employed to measure characteristics that are not tissue stress or strain, but which may have an impact on tissue classification as described herein. Examples of such characteristics include tissue temperature, the distance that needle 102 has been inserted, and tissue electrical properties, e.g., electrical properties of tissue composition 150.

In certain embodiments, probe 104 is formed of a rigid material (e.g., titanium) selected so as to reliably transfer the resistive force from the tissue (e.g., tissue composition 150) to force sensor 110. However, in other embodiments, force sensor 110 and/or position sensor 112 (discussed further herein) may be positioned nearer to the tissue end of the probe (e.g., nearer to end of the probe that makes contact with tissue composition 150) such that less rigid materials may be used for probe 104.

In some embodiments, probe 104 may be configured to apply the mechanical force directionally forward (e.g., such as along axis 103) at local position Pi of local tissue portion 152. In further embodiments, probe 104 may be configured to apply the mechanical force to the tissue composition in a directionally forward direction (e.g., such as along axis 103) beyond distal needle end 102d. For example, in some embodiments, to perform sensing via stimulation of tissue as described herein, the probe may be extended beyond the needle, and possibly retracted back into the needle, by the actuator to apply the mechanical force to tissue.

The needle insertion device 100 may further include a position sensor 112 associated with probe 104. In various embodiments, and as illustrated in the embodiment of FIGS. 1A and 1B, position sensor 112 may be configured to measure an insertion distance Dᵢ, or some portion thereof, of the probe beyond the distal needle end 102d.

The needle insertion device 100 may further include a processor 113 communicatively coupled to force sensor 110 and position sensor 112. In various embodiments, the processor 113 is configured to receive sensor data indicative of the mechanical response to the mechanical force (as provided by force sensor 110) and the insertion distance of probe 104 (as provided by position sensor 112). In still further embodiments, processor 113 may be configured to implement a machine-learning model that, based on the sensor data, predicts a forward distance D_{f} to the remote position Pᵣ of remote tissue portion 154 (e.g., bone). For example, FIG. 1B illustrates the forward distance D_{f} in an embodiment were the distal needle end 102d is inserted into tissue composition 150 along axis 103 at an insertion distance Dᵢ, where Dᵢ may be one or more millimeters in distance.

In various embodiments, the machine-learning model is configured to predict the forward distance to the remote position Pᵣ of the remote tissue portion 154 (e.g., bone) without the distal needle end 102d contacting the remote tissue portion 154. In some such embodiments, the machine-learning model is configured to predict the forward distance to the remote position Pᵣ of the remote tissue portion 154 (e.g., bone) with neither the distal needle end 102d nor the probe 104 contacting the remote tissue portion 154. In other embodiments, the machine-learning model is configured to predict the forward distance to the remote position Pᵣ of the remote tissue portion 154 (e.g., bone) when the probe 104 approaches and nearly touches the remote tissue portion 154. In some embodiments, the forward distance D_{f}, as predicted by the machine-learning model of the needle insertion device 100, is between 2 millimeters and 7 millimeters. In particular embodiments, the forward distance D_{f} is 5 millimeters.

Processor 113 may be used to capture sensor data from force sensor 110 and position sensor 112. The sensor data may be used to run machine-learning based algorithms that classify the tissue near the tip of the needle (e.g., near distal needle end 102d). In some embodiments, as illustrated in FIG. 1A, processor 113 may be incorporated into needle insertion device 100. In other embodiments, needle insertion device 100 may use, in addition to or in the alternative to processor 113, other processor(s) of external devices, such as external devices 130. External devices 130 are external to needle insertion device 100 (e.g., external to casing 120 of needle insertion device 100), and may include devices such as computer 132 or display device 134. Each of the external devices 130 may each include their own processor(s), memory, transceivers (e.g., for sending and receiving sensor data), displays, etc. For example, display device 134 may be, for example, a smart phone or tablet device, such as a device implementing a mobile operating system such as an iPhone or iPad implementing iOS or an Android-based phone or tablet implementing Google's Android platform.

External devices 130 may be communicatively connected to needle insertion device 100 via a wired or wireless connection 131 (e.g., such as via a USB cable or via 802.11 or Bluetooth wireless connection standards) for the purpose of transmitting and/or receiving sensor data. Additionally, or alternatively, sensor data from the needle insertion device 100 may be collected via removable media (e.g., an SD card or similar media device) and processed later.

In various embodiments, the needle insertion device 100 may include a casing 120. In some embodiments, casing 120 may be part of a hand-held embodiment of the needle insertion device 100. Casing 120 may expose buttons and/or data ports (e.g., for USB cable connections or SD cards) for configuration purposes or access to and/or transmission of sensor data as described herein.

In various embodiments, needle insertion device 100 may further include a display 114. In some embodiments, display 114 may implemented as an LCD or LED display screen. In such embodiments, the display screen may be a pixelated screen capable of rendering detailed graphics, charts, or the like. In other embodiments, display 114 may be implemented as a seven-segment display. In other embodiments, display 114 may be an area of the machine-based needle insertion device 100 for display of indicator lights as further described herein.

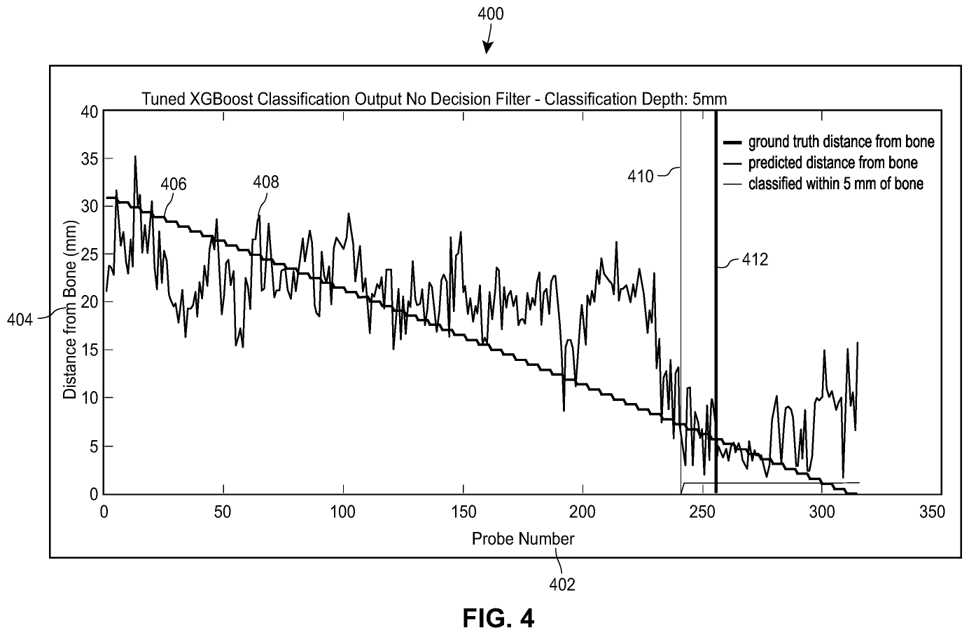

Display 114 may be used for various purposes, including for displaying feedback during insertion of needle 102 into tissue (e.g., tissue composition 150). In some embodiments, for example, as illustrated by FIG. 4, the results of a machine-learning model, including predictions and classifications provided from the machine-learning model, may be provided to a user of the needle insertion device 100 via display 114. Additionally, or alternatively, other information that may be displayed includes providing an estimate of how far the needle is from bone, as described herein.

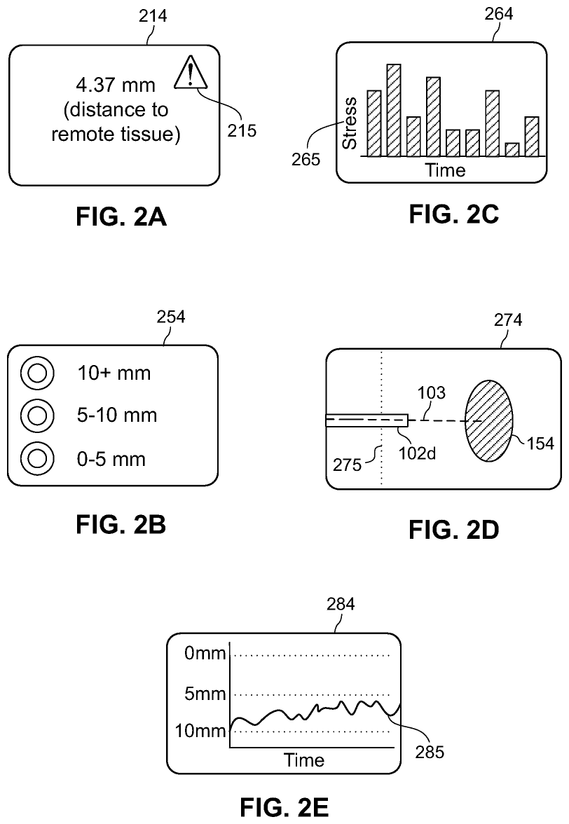

For example, FIG. 2A illustrates a first embodiment of a display 214 associated with the example needle insertion device 100 of FIG. 1A in accordance with various embodiments disclosed herein. FIG. 2A represents an embodiment where display 214 provides an indication of the forward distance D_{f} (e.g., 4.37 mm) to remote position Pᵣ of remote tissue portion 154 (e.g., bone) of tissue composition of 150. Display 214 may be displayed via a display screen, such as via display 114 or an external display of external devices 130 as described herein.

FIG. 2B illustrates a second embodiment of a display 254 associated with the example needle insertion device 100 of FIG. 1A in accordance with various embodiments disclosed herein. In the embodiment of FIG. 2B, display 254 includes one or more indicator light(s). The one or more indicator lights may be implemented as LED lights or other similar lights. For example, the indicator lights of the embodiment of FIG. 2B, may be implemented as indicator light(s) that flash or turn on (e.g., that are switched to an “on” or “lit” state) when needle 102 (e.g., distal needle end 102d) is predicted to within a certain distance from a certain tissue type (e.g., bone). For example, as illustrated, display 254 includes three indicator lights that may flash or turn on as needle 102 (e.g., distal needle end 102d) is predicted, by the machine-learning based model as described herein, to be within 5 mm of bone (i.e., “0-5 mm”), to be between 5 mm and 10 mm of bone (i.e., “5-10 mm”), and/or to be beyond 10 mm of bone (e.g., “10+ mm”). In some embodiments, each of the indicator lights may be of different colors to represent the different distances to bone, (e.g., red, yellow, green to represent the “0-5 mm,” “5-10 mm,” and “10+ mm” distances illustrated in FIG. 2A, respectively). In embodiments where indicator lights are physical lights, display 254 may be included as part of needle insertion device 100, such as positioned on needle insertion device 100 as display 114 is shown in FIG. 1A. Additionally, or alternatively, in embodiments where indicator lights are implemented as graphical lights, display 254 may be implemented as a display screen, such as a display screen rendered via display 114 or via an external display of external devices 130 as described herein.

FIG. 2C illustrates a third embodiment of a display 264 associated with the example needle insertion device of FIG. 1A in accordance with various embodiments disclosed herein. In particular, display 264 illustrates pre-processed sensor data showing stress levels 265 over time as experienced by tissue (e.g., tissue composition 150) in contact with probe 104. In such embodiments, stress levels 265 may be measured using a Fast Fourier transform (FFT) algorithm, e.g., derived mechanical responses (e.g., viscoelastic responses), and/or other transformations of the sensor data received by sensors 110 and/or 112. Display 264 may be useful for a user of the needle insertion device 100 for troubleshooting purposes. Display 264 may be displayed via a display screen, such as via display 114 or an external display of external devices 130 as described herein.

FIG. 2D illustrates a fourth embodiment of a display 274 associated with the example needle insertion device 100 of FIG. 1A in accordance with various embodiments disclosed herein. In particular, display 274 illustrates a diagram showing a representation of needle 102 (e.g., distal needle end 102d) distance from remote tissue portion 154 (e.g., bone) along axis 103 as described herein. That is, the diagram may represent, graphically, needle 102's predicted distance from tissue portion 154 (e.g., bone). The diagram of display 274 may also include a classification distance indicator line 275, illustrating needle 102's forward distance to tissue portion 154 (e.g., bone) as further described herein. Display 274 may be displayed via a display screen, such as via display 114 or an external display of external devices 130 as described herein.

FIG. 2E illustrates a fifth embodiment of a display 284 associated with the example needle insertion device of FIG. 1A in accordance with various embodiments disclosed herein. In particular, display 284 shows an estimated distance history 285 plotted over time that illustrates distance estimates for past probe events, as further described herein. Such past probe events and estimated distance history may be useful in showing a user of the needle insertion device 100 a longer term view of needle 102's approach into tissue. Such information may provide the user with an indication as to whether needle insertion is different from a normal or expected approach, for example, where a particular patient's tissue is being more or less resistive than compared to average patients. Display 284 may be displayed via a display screen, such as via display 114 or an external display of external devices 130 as described herein.

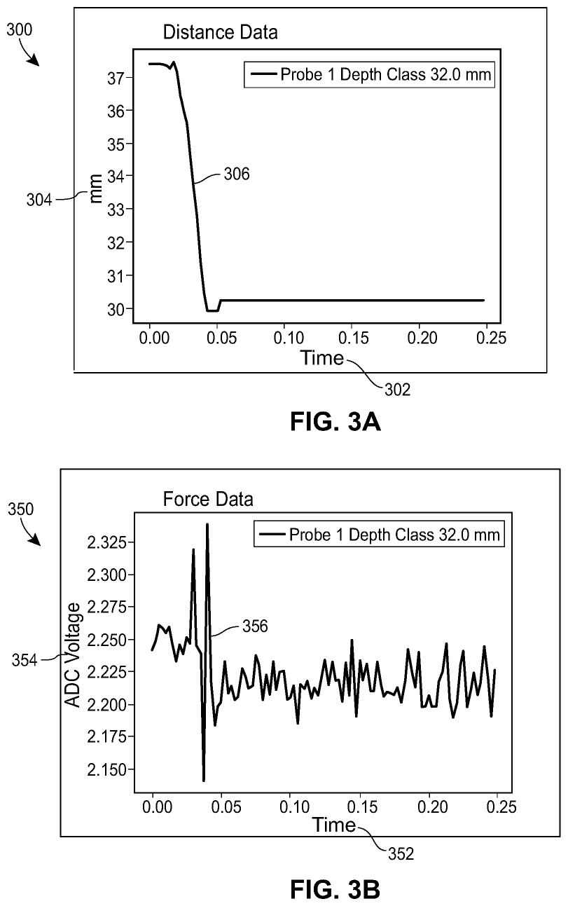

In some embodiments, such as the embodiments of FIG. 1A, display 114 is incorporated within, or partially within, casing 120 of needle insertion device 100. In other embodiments, however, the displays of FIGS. 2A-2E, or other displays, figures, or screens as described herein (e.g., as illustrated via FIGS. 3A, 3B, 4, and 5), may be implemented on displays external to the needle insertion device 100. For example, as illustrated in FIG. 1A, external devices 130 include display screen on which any of the displays described herein may be implemented. Such displays may be implemented via an application (app), pop-up window, or other software rendered screen of computer 132 or display device 134, or other such similar devices.

In further embodiments, and regardless of whether display 114 is included as part of needle insertion device 100, or external to it, display 114 may be configured to provide an alert indicating that needle 102 (e.g., distal needle end 102d) is within a threshold distance from remote tissue portion 154. For example, in the embodiment of FIG. 2A, alert 215 is provided to indicate that needle 102 (e.g., distal needle end 102d) is predicted to be within 4.37 mm (i.e., a threshold distance) of remote tissue portion 154 (e.g., bone). Alerts may also be similarly provided for the displays of the embodiments illustrated in each of FIGS. 2B-2E.

Additionally, or alternatively, in further embodiments, auditory alerts may also be provided by needle insertion device 100. Such auditory alerts may be triggered as described above, but where a speaker or other auditory device (not shown) of needle insertion device 100 is activated to inform a user that needle 102 (e.g., distal needle end 102d) is predicted to be within a threshold distance of remote tissue portion 154 (e.g., bone). In some embodiments, a tone or pitch of the auditory alert or signal may be varied with the distance from the remote tissue portion 154 (e.g., bone) to indicate distance to the user with audible feedback.

Description of Sensing Techniques

As described herein, needle insertion device(s), including, for example, any of a predictive and/or machine-learning based needle insertion device(s), may utilize mechanical stimulation of tissue (e.g., tissue composition 150) in order to measure a mechanical response from such tissue. For example, the mechanical stimulation may be applied as a mechanical force by probe 104 to local tissue portion 152 and/or remote tissue portion 154 of tissue composition 150. When force (e.g., a mechanical force) is applied, the tissue may exhibit a resistive force which may be measured as a force response (e.g., a mechanical response) by force sensor 110.

In some embodiments, the mechanical response may change as the mechanical force is applied across different frequencies that generate frequency-dependent tissue responses. Such frequency-dependent tissue responses include force responses at different frequencies, and may vary with tissue type (e.g., vary based on whether the tissue type is local tissue portion 152, such as soft tissue, or remote tissue portion 154, such as bone). The different response frequencies can be used to train and implement predictive and/or machine-learning based classification and/or prediction model(s), such as those described herein.

The different response frequencies may be obtained using various techniques, where, for example, respective mechanical force(s) are applied via various embodiments, and the different response frequencies are sensed by force sensor 110. For example, in various embodiments a mechanical force may be implemented as one of a plurality of multi-frequency forces, in which the different response frequencies are respectively obtained. In such embodiments, actuator 106 may be operable to actuate probe 104 periodically in order to apply the plurality of multi-frequency forces during a corresponding plurality of actuation iterations. For example, in certain embodiments, the each of the plurality of actuation iterations may include probe 104 extending and retracting along an axis, such as axis 103 as illustrated in in the embodiment of FIGS. 1A and 1B.

In some embodiments, each of the plurality of frequencies of the plurality of multi-frequency forces is determined via step-input actuation. In such embodiments, the step-input actuation may be based on a sinusoidal signal provided to the actuator. For example, the sinusoidal signal may be based on a step function implemented by the process of the needle insertion device 100. Generally, a step function is a sum of a series of pure sinusoidal signals with widely varying frequencies. Measuring stress and strain of tissue (e.g., tissue composition 150) during application of probe 104 via actuator 106, as described herein, via a mechanical step function allows characterization and/or classification of frequency-dependent tissue response. Such characterization and/or classification can be used to train and implement predictive and/or machine-learning based classification and/or prediction model(s), such as those described herein.

Additionally, or alternatively, the plurality of frequencies of the plurality of multi-frequency forces may be determined via varied sinusoidal frequencies provided to the actuator. For example, tissue force response may also be sensed using sinusoids with varying frequencies to obtain sensor data for tissue classification. Other dynamic input waveforms may also be substituted in place of the varied sinusoidal frequencies. For example, a chirped sinusoid could be used to improve signal to noise ratio and/or to sense tissue force response(s) by varying frequencies to obtain sensor data for tissue classification.

In other embodiments, zero-frequency data collection may be used. In such embodiments, sensor data is observed and collected for long time scale near-steady-state response(s). For example, in such embodiments, a mechanical force may be one of a plurality of forces applied to the tissue composition 150 and the resistive force may be one of a near-steady-state response received during a zero-frequency data collection where probe 104 is actuated over long time scale observation.

Description of Machine-Learning Based Classification and Prediction Model

In various embodiments described herein, a machine-learning model may determine how far needle 102 (e.g., distal needle end 102d) is from certain types of tissue (e.g., bone, as represented, for example, by remote tissue portion 154). The machine-learning model takes as input sensor data, e.g., probe force and distance data from force sensor 110 and distance and position sensor 112, respectively, and outputs the predicted distance from the tissue type (e.g., bone).

Generally, machine-learning models, as described herein, may be trained using a supervised or unsupervised machine-learning program or algorithm. The machine-learning program or algorithm may employ a neural network, which may be a convolutional neural network, a deep learning neural network, or a combined learning module or program that learns based on one or more features or feature datasets in particular areas of interest. The machine-learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine-learning algorithms and/or techniques. Machine learning, as described herein, may the particular machine-learning algorithm (e.g., a neural network algorithm) identifying and recognizing patterns in existing data (e.g., such as patterns in sensor data provided by force sensor 110 and distance and position sensor 112) in order to facilitate making predictions and/or classifications for subsequent data (e.g., to predict and/or classify a forward distance to remote position Pᵣ of remote tissue portion 154).

Machine-learning model(s), such as those described herein as utilized with needle insertion device 100, may be created and trained based upon example (e.g., “training data,”) inputs or data (which may be termed “features” and “labels”) in order to make valid and reliable predictions for new inputs, such as testing level or production level data or inputs. In supervised machine-learning, a machine-learning program operating on a server, computing device, or otherwise processor(s), may be provided with example inputs (e.g., “features”) and their associated, or observed, outputs (e.g., “labels”) in order for the machine-learning program or algorithm to determine or discover rules, relationships, or otherwise machine-learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict or classify, based on the discovered rules, relationships, or model, an expected output.

In unsupervised machine-learning, the server, computing device, or otherwise processor(s), may be required to find its own structure in unlabeled example inputs, where, for example, multiple training iterations are executed by the server, computing device, or otherwise processor(s) to train multiple generations of models (e.g., new models) until a satisfactory model, e.g., a model that provides sufficient prediction accuracy when given test level or production level data or inputs, is generated. The disclosures herein may use one or both of such supervised or unsupervised machine-learning techniques.

In various embodiments a machine-learning model, as utilized by the needle insertion device 100, is a classifier based machine-learning model. Such classifier based machine-learning models may be, or include, a classifier for making predictions where a decision is classified as one type from a plurality of available types (e.g., whether tissue is of one type or another). In such embodiments, the sensor data (e.g., as provided by force sensor 110 and distance and position sensor 112) is input to the machine-learning model to determine a corresponding machine-learning based classification. For example, the machine-learning based classification may generate a classification that may include a local classification indicating, and corresponding to, local tissue portion 152 (e.g., soft tissue). Similarly, the machine-learning based classification may include a remote classification indicating, and corresponding to, remote tissue portion 154 (e.g., bone). Such classifications generally indicate that the machine-learning model, based on the sensor data, has classified a particular detected tissue type as one type or the other (e.g., as local tissue portion 152 or remote tissue portion 154).

In some embodiments, the machine-learning model may be based on a recurrent neural network algorithm. In other embodiments, the machine-learning model may be based on a tree-based retrogression algorithm. In still further embodiments, the machine-learning model may include, or apply, a depth-sensitive average (DSA) filtering as described herein.

For training machine-learning models, sensor data (e.g., as provided by force sensor 110 and distance and position sensor 112) may be collected on sample tissue sets (e.g., cadaver tissue or tissue similar to human tissue, e.g., pig tissue). Such sensor data may be obtained at different needle depths, e.g., by inserting and advancing needle 102 and probing, with probe 104, a tissue sample until reaching a particular position within the tissue. For example, in the embodiment shown in FIGS. 1A and 1B, the particular position may be Pᵣ of tissue composition 150, which may represent striking bone (e.g., remote tissue portion 154). In one embodiment, each insertion into the tissue until bone is struck may constitute an “approach,” where each approach may include several probe events. A single probe event may correspond to one or more feature(s), or feature vector(s), which are used to train the machine-learning model. The feature(s) in each probe event may include the raw probe force data and/or distance data (e.g., as determined from sensor data provided by force sensor 110 and distance and position sensor 112) as well as statistical values such as means and standard deviations of subsequences of that data. Machine-learning labels may also be generated for each probe event for each approach indicating how far needle 102 was from bone (e.g., remote tissue portion 154). Together, the labels and feature(s) may be used to train the machine-learning model of the needle insertion device 100 as described herein.

Examples of sensor data, which may be used as feature(s) for training machine-learning models, is illustrated in FIGS. 3A and 3B. Each of FIGS. 3A and 3B show sensor data as captured over simultaneous time sequences during a time period where actuator 106 was in an actuating state with respect to a tissue sample. FIG. 3A illustrates an example display 300 of sensor data indicative of insertion distance (e.g., as sensed by position sensor 112) of probe 104 of the example needle insertion device 100 of FIG. 1A in accordance with various embodiments disclosed herein. The sensor data of FIG. 3A represents a single probe event, and includes a hundred data points plotted (306) across distance axis 304 (showing insertion distance in millimeters) and time axis 302 (showing time in fractions of a second). Display 300 may be displayed via a display screen, such as via display 114 or an external display of external devices 130 as described herein.

FIG. 3B illustrates an example display 350 of sensor data indicative of a mechanical response (e.g., as sensed by force sensor 110) to a mechanical force applied by probe 104 of the example needle insertion device 100 of FIG. 1A in accordance with various embodiments disclosed herein. As for the sensor data of FIG. 3A, the sensor data of FIG. 3B represents a single probe event, and includes a hundred data points plotted (356) across ADC (Analog-to-Digital Converter) Voltage axis 354 (showing voltage) and time axis 352 (showing time in fractions of a second). ADC voltage may be a unit measured by force sensor 110 and is representative of the mechanical response and/or resistive force as describe herein. Display 350 may be displayed via a display screen, such as via display 114 or an external display of external devices 130 as described herein.

Using the sensor data (e.g., as shown in FIGS. 3A and 3B) as features, and pairing it with the machine-learning labels as described above, the machine-learning may be trained for use in predicting and/or classifying how far away a single probe event is from a particular tissue type (e.g., bone). Such prediction/classification provides an indication of how far away needle 102 (e.g., distal needle end 102d) is from bone, e.g., during an epidural placement or other medical procedure.

Once the machine-learning model is trained, the machine-learning model may be used in a needle insertion device 100 as described herein. Computer Program Listing 1 below shows pseudo code for how a machine-learning model may be implemented with a needle insertion device 100.

Computer Program Listing 1

#List for holding predicted bone distances. Initialized to empty.
predicted_bone_distance_list = [ ]
while user is advancing needle:

# Probe tissue periodically
wait(time_between_probes)
# Actuate probe and collect data
start_recording( )
actuate_probe( )
end_recording( )
data = get_recorded_data( )
# Have classifier predict distance from bone based on data
# Preprocess data
pre_processed_data = pre_process(data)
# Get bone depth prediction
predicted_bone_distance = classifier(pre_processed_data)
# Add predicted distance to list
predicted_bone_distance_list.append(predicted_bone_distance)
# Perform filtering based on current and past predictions
filtered_bone_distance = filter(predicted_bone_distance_list)
# Give feedback to user. Two options shown.
# START Option 1: Display prediction on feedback device to user in
real-
# time.
display_prediction(filtered_bone_distance)
# END Option 1
# START Option 2: Alert user if the needle is within a certain
distance
# from bone.
if filtered_bone_distance < classification_distance:
alert_user( )
end if
# END Option 2

end while

As illustrated by the pseudo code of Computer Program Listing 1, first, the machine-learning classifier function (classifier( )) is trained before the algorithm of Computer Program Listing 1 runs. However, in some embodiments, the trained model could be retrained using data collected during the procedure. For example, in some embodiments, a new machine-learning model may be trained with received sensor data (e.g., as provided by force sensor 110 and distance and position sensor 112). In such embodiments, an existing machine-learning model may be updated with the new machine-learning model that is based on the newly provided sensor data.

Second, the algorithm of Computer Program Listing 1 shows data preprocessing as well as data filtering, which are both optional. The data filtering allows the algorithm to incorporate past probe events into its decision for a current probe event. In some embodiments, the data filtering may be incorporated into the classifier itself, e.g., where the classifier is a recurrent neural network that makes decisions based on multiple and/or recurrent probe events.

Third, probing, via probe 104, may be done periodically based various factors, including based on time (e.g., a given number of probes per second), needle insertion depth (e.g., execute a probe event every time the needle advances a given distance measured in millimeters), user input (e.g., execute a probe event when the user pushes a button), or other factors including combinations of those already described herein. For example, the pseudo code of Computer Program Listing 1 executes probe events based on time.

Fourth, the needle insertion device 100 may give a user one or more different forms of feedback. For example, such feedback may be displayed by display 114 (or external devices 130) as described herein. In the embodiment of Computer Program Listing 1, two options are illustrated, including displaying an estimated distance to bone after each probe event (e.g., such as illustrated by FIG. 2A) and alerting the user once the needle is within a certain distance (classification distance) from bone (e.g., also as illustrated by FIG. 2A).

Based on the feedback provided to the user, as illustrated by Computer Program Listing 1, the user can adjust the position of needle 102 (e.g., distal needle end 102d) and its angle within the tissue of a patient in order to successfully steer or position needle 102 during performance of a medical procedure. In addition, for an epidural procedure, and in one embodiment, once the user is confident that the epidural space has been located, probe 104, and possibly the sensing hardware (e.g., force sensor 110, position sensor 112, etc.) may be removed, thus allowing the user to confirm needle placement using standard techniques, e.g., the loss of resistance technique, and to insert or thread the catheter in order to administer fluid, e.g., anesthesia.

Additional Machine-Learning Models and Considerations

Various types of machine-learning models may be used with needle insertion device 100, as described herein. For example, the machine-learning based labels and feature(s) as described above may be used to train various machine learning models based on various respective algorithms. For example, in one example embodiment the machine-learning based labels and feature(s) as described above may be used to train a tree-based regression algorithm referred to as “XGBOOST.” The XGBOOST algorithm takes as input an individual probe event and outputs the predicted distance from a tissue type (e.g., bone) for that probe event. The trained XGBOOST algorithm is analogous to the classifier that appears in Computer Program Listing 1 as described herein. In particular, the XGBOOST algorithm may be used to train a machine-learning model that predicts when needle 102 is within a certain distance (e.g., a forward distance) from a certain tissue type (e.g., bone). Such prediction may be used to alert a user of the needle insertion device 100 when the forward distance is with a certain distance or threshold of the tissue type as described herein. It is to be understood that XGBOOST algorithm is one of several algorithms that may be used to train machine learning models that may be used with for needle insertion device 100 as described herein. Needle insertion device 100 does not rely on any particular machine learning model implementation or related training thereof, and other machine learning models and/or training may be used in accordance with the disclosure herein.

A classifier of an XGBOOST based machine-learning model may be trained on sensor data collected across multiple needle approaches and tested on sensor data taken from one or more needle approaches. For example, in one embodiment, once the XGBOOST classifier is trained, test data points (i.e., sensor data used as data to test the XGBOOST based machine-learning model) may be fed into the XGBOOST classifier one data point at a time starting with data representing the approach of needle 102 furthest from bone and ending with the approach at which bone is struck. Such an approach simulates the order of sensor data generally experienced as the needle is inserted into tissue (e.g., tissue composition 150). The outputs of the classifier may be fed into a decision function that determines the first point at which the needle is within a certain distance from bone. In some embodiments, such distance may be referred to as the “classification distance” (e.g., which, in some embodiments, may be a forward distance as described herein) and the identified position of the probe may be referred to as the “intercept point” (e.g., which, in some embodiments, may local position Pi as describe herein). For example, if the classification distance is 5 mm, then the first probe event for which the XGBOOST classifier predicts a value of 5 mm or less is taken as the intercept point. In such embodiments, the needle insertion device 100 would use the XGBOOST based machine-learning model to alert the user that the needle is near bone, as illustrated in FIG. 2A, and as demonstrated in feedback Option 2 of Computer Program Listing 1.

In some embodiments, decision functions (analogous to the filter function in Computer Program Listing 1) are used as part of, or in addition to, machine-learning model as described herein. For example, in some embodiments, depth-sensitive average (DSA) filtering is utilized in addition to a machine-learning model. For example, for a machine-learning model based on the XGBOOST algorithm, DSA filtering averages the XGBOOST classifier output for up to the last three data points having values of one millimeter from each other. It is to be understood, however, that other embodiments do not apply filtering. Also, it is to be understood that depth-sensitive averaging can be implemented to have different limits of the number of points and/or distance(s) between such points. In particular, more possibilities than the three most recent points within one mm of each other are contemplated herein. For example, additional or fewer points with different various distances are contemplated herein.

FIG. 4 illustrates an example display 400 of predictions and classifications regarding a tissue composition (e.g., tissue composition 150) as associated with the example needle insertion device 100 of FIG. 1A in accordance with various embodiments disclosed herein. Display 400 may be displayed via a display screen, such as via display 114 or an external display of external devices 130 as described herein.

FIG. 4 illustrates the display output of an XGBOOST classifier and its decision function for a needle approach into a tissue composition. In the embodiment of FIG. 4, classification for a needle approach includes a classification distance of 5 mm. Each point in display 400 corresponds to a probe event associated with probe number axis 402 and probe depth axis 404, where the multiple probe events are taken at various distances from bone (in millimeters). Line 406 illustrates an actual distance (i.e., a ground truth distance) from bone of each probe event. Line 408 illustrates the prediction output of the XGBOOST machine-learning model. Line 410 illustrates the intercept point (e.g., local position Pi). Line 412 indicates an actual first time when the needle is within 5 mm of bone.

In various embodiments, error data or statistical data may be determined to evaluate the performance of machine-learning model(s) as used with the needle insertion device 100 as described herein. In some embodiments, the error for an individual test may be determined as the difference between the classification distance and the distance from bone of the intercept point (e.g., the depth of the probe at line 412 minus the depth of the probe at line 410 as illustrated in FIG. 4). Cross-validation may be used to obtain error values for each of a plurality of recorded needle approaches (as described for FIG. 4). Using the error values, each of an average error, an average absolute error, and a root mean squared error (RMSE) may be determined. The ideal value for each of these error values is zero, where a zero error indicates that the classification or prediction of the machine-learning model is completely accurate. An example set of error values for a classification distance of 5 mm using an XGBOOST based machine-learning model is illustrated below in Table 1.

TABLE 1
(XGBOOST Results - Classification Distance: 5 mm)

| Avg. Absolute Error | Avg. Error | Root Mean Square Error |
|---|---|---|
| 2.96428571 | 1.39285714 | 3.33541602 |

The error values of Table 1 are determined from a XGBOOST machine-learning model, trained with sensor data as described herein, and using DSA filtering. The error values of Table 1 show that the average error is approximately 1.39 mm, which illustrates that, on average, the XGBOOST machine-learning model, for this particular embodiment, predicts that the needle is 5 mm away from bone when it is actually 3.61 mm away from bone. The average absolute error and root mean square (RMSE) values each include approximately 3 mm of variation. Because both the average absolute error and the RMSE are of similar values (i.e., approximately 3 mm each), in this embodiment, the similar 3 mm values indicate a variation that is spread across most needle approaches as opposed to concentrated in a few outliers. It is to be understood that error values in Table 1 represent a single embodiment of example error values, and that other error values are contemplated herein.

In further embodiments, such error values may be reduced through refinement of the classifier of XGBOOST machine-learning model via the collection of additional sensor data and retraining of the XGBOOST machine-learning model. For example, this may be achieved via the training of a new XGBOOST machine-learning model with additional sensor data as described herein.

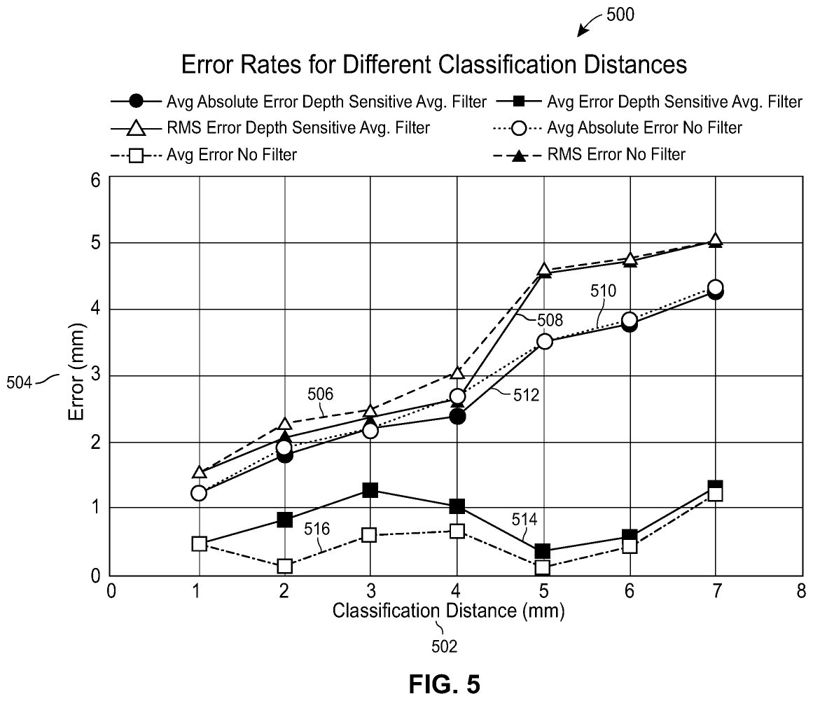

FIG. 5 illustrates an example display 500 showing error values for different classifications as associated with the example needle insertion device of FIG. 1A in accordance with various embodiments disclosed herein. Display 500 may be displayed via a display screen, such as via display 114 or an external display of external devices 130 as described herein.

In particular, FIG. 5 shows error values for different classification distances. The three error metrics as illustrated for Table 1 (i.e., avg. absolute error, avg. error, and root mean square error) are each determined for different classification depths. Generally, dashed error lines (lines 506, 510, and 516) correspond to the error metrics for when no decision filter (e.g., no DSA filter) is applied to the raw XGBOOST classifier results. In contrast, solid error lines (lines 508, 512, and 514) correspond to the metrics for when a DSA filter is applied. As shown by classification distance axis 502, classification distances range from 1 mm to 7 mm. As shown by error value axis 504, error values range from 0 mm to 5 mm. Other embodiments may include different or additional distances and/or error value ranges.

Error lines 506-516 of FIG. 5 represent error values for the XGBOOST machine-learning model for different classification distances. Each error line 506-516 represents a different configuration or implementation of the XGBOOST machine-learning model, which explains the difference in error values across each of the error lines 506-516. As described herein, error lines 506, 510, and 516 represent error results for when no decision function (e.g., DSA filter) is applied, where error line 506 represents the RMS error (RSME) when no DSA filtering is applied, error line 510 represents the average absolute error when no DSA filtering is applied, and error line 516 represents the average error when no DSA filtering is applied.

Error lines 508, 512, and 514 represent error results for when a DSA filter is applied, where error line 508 represents the RMS error (RSME) when DSA filtering is applied, error line 512 represents the average absolute error when DSA filtering is applied, and error line 514 represents the average error when DSA filtering is applied.

As illustrated in FIG. 5, slight differences may occur between error values for when the DSA filter is applied and when no filter is applied, at least for some of the error data. For example, error values associated with DSA filtering generally demonstrate better results (lower error) for absolute average error and root mean square error, but demonstrate worse results (higher error) for average error. Implementations of the XGBOOST machine-learning model using DSA filtering is generally preferred for avoiding spurious spikes in XGBOOST machine-learning model's prediction/classifier decision. However, models using DSA filtering may experience a slight delay when a true classification depth is found.

In addition, as shown in FIG. 5, error values generally decrease for smaller classification distances, with a large drop-off in RMSE and absolute average error from 5 mm to 4 mm. Thus, in some embodiments, the results shown in FIG. 5, and Table 1, may be improved if the classification distance were set to 4 mm rather than 5 mm. The error values illustrated in the embodiment of FIG. 5, may be used to retrain new machine-learning model(s) for use with the needle insertion device 100 as described herein. For example, by reviewing the error values of FIG. 5, a user may determine to train new machine-learning model, as described herein, having improved accuracy where the predicted classification distance, as output by the new machine-learning model, may be reduced in error thus providing more accurate feedback (e.g., via display 114) to a user of the needle insertion device 100.

### Aspects of the Disclosure

1. A predictive needle insertion device comprising: a needle having a proximal needle end and a distal needle end; a probe movably coupled to the needle, the probe movable to a position extending beyond the distal needle end; an actuator operable to actuate the probe to apply a mechanical force to a tissue composition, wherein the tissue composition includes a local tissue portion and a remote tissue portion, the local tissue portion being at a local position to the distal needle end and the remote tissue portion being at a remote position to the distal needle end; a force sensor associated with the probe, the force sensor configured to detect a mechanical response to the mechanical force, the mechanical response being indicative of a resistive force; a position sensor associated with the probe, the position sensor configured to measure an insertion distance of the probe beyond the distal needle end; a processor communicatively coupled to the force sensor and the position sensor, the processor configured to receive sensor data indicative of the mechanical response to the mechanical force and the insertion distance of the probe; and a non-transitory program memory communicatively coupled to the processor and storing executable instructions that, when executed by the processor, cause the processor to predict, based on the sensor data, a forward distance to the remote position of the remote tissue portion.

2. The predictive needle insertion device of aspect 1, wherein the remote tissue portion is bone.

3. The predictive needle insertion device of any of the aforementioned aspects, wherein the processor, executing the instructions, predicts the forward distance to the remote position of the remote tissue portion without the distal needle end contacting the remote tissue portion.

4. The predictive needle insertion device of any of the aforementioned aspects, wherein the probe is configured to apply the mechanical force directionally forward at the local position of the local tissue portion.

5. The predictive needle insertion device of any of the aforementioned aspects, wherein the probe is configured to apply the mechanical force to the tissue composition directionally forward beyond the distal needle end.

6. The predictive needle insertion device of any of the aforementioned aspects, wherein the mechanical force is one of a plurality of multi-frequency forces, and wherein the actuator is further operable to actuate the probe periodically to apply the plurality of multi-frequency forces during a corresponding plurality of actuation iterations.

7. The predictive needle insertion device of aspect 6, wherein each of the plurality of actuation iterations includes the probe extending and retracting along an axis associated with the needle.

8. The predictive needle insertion device of aspect 6, wherein a plurality of frequencies of the plurality of multi-frequency forces is determined via step-input actuation.

9. The predictive needle insertion device of aspect 8, wherein the step-input actuation is based on a sinusoidal signal provided to the actuator.

10. The predictive needle insertion device of aspect 6, wherein a plurality of frequencies of the plurality of multi-frequency forces is determined via varied sinusoidal frequencies provided to the actuator.

11. The predictive needle insertion device of any of the aforementioned aspects, wherein the mechanical force is one of a plurality of forces applied to the tissue composition and the resistive force is a near-steady-state response received during a zero-frequency data collection actuation of the probe over long time scale observation.

12. The predictive needle insertion device of any of the aforementioned aspects, wherein the probe is capable of retracting into the distal needle end.

13. The predictive needle insertion device of any of the aforementioned aspects, wherein the probe is an inter-needle probe operable to extend through the proximal needle end and the distal needle end.

14. The predictive needle insertion device of any of the aforementioned aspects, wherein the needle is a 17 gauge needle.

15. The predictive needle insertion device of any of the aforementioned aspects, wherein the needle is a disposable needle and the probe is a disposable probe, wherein each of the disposable needle and the disposable probe are removably coupled to the predictive needle insertion device.

16. The predictive needle insertion device of any of the aforementioned aspects, wherein the needle is operable to receive a catheter through the proximal needle end and the distal needle end.

17. The predictive needle insertion device of any of the aforementioned aspects, further comprising a display.

18. The predictive needle insertion device of aspect 17, wherein the display includes an indicator light.

19. The predictive needle insertion device of aspect 17, wherein the display includes a display screen.

20. The predictive needle insertion device of aspect 17, wherein the display provides an indication of the forward distance to the remote position of the remote tissue portion.

21. The predictive needle insertion device of aspect 17, wherein the display provides an alert indicating that the distal needle end is within a threshold distance from the remote tissue portion.

22. The predictive needle insertion device of any of the aforementioned aspects, wherein the processor is an external processor external to a casing of the predictive needle insertion device.

23. The predictive needle insertion device of aspect 22, wherein the external processor receives the sensor data via wireless communication.

24. A machine-learning based needle insertion device comprising: a needle having a proximal needle end and a distal needle end; a probe movably coupled to the needle, the probe capable of extending beyond the distal needle end; an actuator operable to actuate the probe to apply a mechanical force to a tissue composition, wherein the tissue composition includes a local tissue portion and a remote tissue portion, the local tissue portion being at a local position to the distal needle end and the remote tissue portion being at a remote position to the distal needle end; a force sensor associated with the probe, the force sensor configured to determine a resistive force of the tissue composition, the resistive force measured as a mechanical response to the mechanical force; a position sensor associated with the probe, the position sensor configured to measure an insertion distance of the probe beyond the distal needle end; and a processor communicatively coupled to the force sensor and the position sensor, the processor configured to receive sensor data indicative of the mechanical response to the mechanical force and the insertion distance of the probe, and the processor further configured to implement a machine-learning model that, based on the sensor data, predicts a forward distance to the remote position of the remote tissue portion.

25. The machine-learning based needle insertion device of aspect 24, wherein the remote tissue portion is bone.

26. The machine-learning based needle insertion device of any one or more of aspects 24 to 25, wherein the machine-learning model predicts the forward distance to the remote position of the remote tissue portion without the distal needle end contacting the remote tissue portion.

27. The machine-learning based needle insertion device of any one or more of aspects 24 to 26, wherein the probe is configured to apply the mechanical force directionally forward at the local position of the local tissue portion.

28. The machine-learning based needle insertion device of any one or more of aspects 24 to 27, wherein the probe is configured to apply the mechanical force directionally forward to the tissue composition beyond the distal needle end.

29. The machine-learning based needle insertion device of any one or more of aspects 24 to 28, wherein the mechanical force is one of a plurality of multi-frequency forces, and wherein the actuator is further operable to actuate the probe periodically to apply the plurality of multi-frequency forces during a corresponding plurality of actuation iterations.

30. The machine-learning based needle insertion device of aspect 29, wherein each of the plurality of actuation iterations incudes the probe extending and retracting along an axis associated with the needle.

31. The machine-learning based needle insertion device of aspect 29, wherein a plurality of frequencies of the plurality of multi-frequency forces is determined via step-input actuation.

32. The machine-learning based needle insertion device of aspect 31, wherein the step-input actuation is based on a sinusoidal signal provided to the actuator.

33. The machine-learning based needle insertion device of aspect 29, wherein a plurality of frequencies of the plurality of multi-frequency forces is determined via varied sinusoidal frequencies provided to the actuator.

34. The machine-learning based needle insertion device of any one or more of aspects 24 to 33, wherein the mechanical force is one of a plurality of forces applied to the tissue composition and the resistive force is a near-steady-state response received during a zero-frequency data collection actuation of the probe over long time scale observation.

35. The machine-learning based needle insertion device of any one or more of aspects 24 to 34, wherein the probe capable of retracting into the distal needle end.

36. The machine-learning based needle insertion device of any one or more of aspects 24 to 35, wherein the probe is an inter-needle probe operable to extend through the proximal needle end and the distal needle end.

37. The machine-learning based needle insertion device of any one or more of aspects 24 to 36, wherein the needle is a 17 gauge needle.

38. The machine-learning based needle insertion device of any one or more of aspects 24 to 37, wherein the needle is a disposable needle and the probe is a disposable probe, wherein each of the disposable needle and the disposable probe are removably coupled to the machine-learning based needle insertion device.

39. The machine-learning based needle insertion device of any one or more of aspects 24 to 38, wherein the needle is operable to receive a catheter through the proximal needle end and the distal needle end.

40. The machine-learning based needle insertion device of any one or more of aspects 24 to 39, further comprising a display.

41. The machine-learning based needle insertion device of aspect 40, wherein the display includes an indicator light.

42. The machine-learning based needle insertion device of aspect 40, wherein the display includes a display screen.

43. The machine-learning based needle insertion device of aspect 40, wherein the display provides an indication of the forward distance to the remote position of the remote tissue portion.

44. The machine-learning based needle insertion device of aspect 40, wherein the display provides an alert indicating that the distal needle end is within a threshold distance from the remote tissue portion.

45. The machine-learning based needle insertion device of any one or more of aspects 24 to 44, wherein the processor is an external processor external to a casing of the machine-learning based needle insertion device.

46. The machine-learning based needle insertion device of aspect 45, wherein the external processor receives the sensor data via wireless communication.

47. The machine-learning based needle insertion device of any one or more of aspects 24 to 46, wherein the machine-learning model is a classifier based machine-learning model, and wherein the sensor data is input to the machine-learning model to determine a corresponding machine-learning based classification, wherein the machine-learning based classification may include one of a local classification corresponding to the local tissue portion or a remote classification corresponding to the remote tissue portion.

48. The machine-learning based needle insertion device of aspect 47, wherein the machine-learning model is based on a recurrent neural network algorithm.

49. The machine-learning based needle insertion device of aspect 47, wherein the machine-learning model is based on a tree-based retrogression algorithm.

50. The machine-learning based needle insertion device of aspect 47, wherein the machine-learning model applies depth-sensitive average filtering.

51. The machine-learning based needle insertion device of any one or more of aspects 24 to 50, wherein a new machine-learning model is trained with the received sensor data.

52. The machine-learning based needle insertion device of aspect 51, wherein the machine-learning model is updated with the new machine-learning model.

53. A method for utilizing a predictive needle insertion device during a medical procedure, the method comprising: inserting a needle into a tissue composition, the needle having a proximal needle end and a distal needle end; applying, by a probe movably coupled to the needle and movable to a position extending beyond the distal needle end, a mechanical force to the tissue composition, wherein the tissue composition includes a local tissue portion and a remote tissue portion, the local tissue portion being at a local position to the distal needle end and the remote tissue portion being at a remote position to the distal needle end; detecting, with a force sensor associated with the probe, a mechanical response to the mechanical force, the mechanical response being indicative of a resistive force; measuring, with a position sensor associated with the probe, an insertion distance of the probe beyond the distal needle end; receiving, by a processor communicatively coupled to the force sensor and the position sensor, sensor data indicative of the mechanical response to the mechanical force and the insertion distance of the probe; and predicting, with the processor based on the sensor data, a forward distance to the remote position of the remote tissue portion.

54. A tangible, non-transitory computer-readable medium storing instructions, that when executed by one or more processors of a predictive needle insertion device cause the one or more processors of the predictive needle insertion device to: detect, with a force sensor associated with a probe movably coupled to a needle having a proximal needle end and a distal needle end, a mechanical response to a mechanical force, the mechanical response being indicative of a resistive force, wherein the probe is movable to a position extending beyond the distal needle end, and wherein the probe is configured to apply the mechanical force to a tissue composition, wherein the tissue composition includes a local tissue portion and a remote tissue portion, the local tissue portion being at a local position to the distal needle end and the remote tissue portion being at a remote position to the distal needle end; measure, with a position sensor associated with the probe, an insertion distance of the probe beyond the distal needle end; receive, by a processor communicatively coupled to the force sensor and the position sensor, sensor data indicative of the mechanical response to the mechanical force and the insertion distance of the probe; and predict, with the processor based on the sensor data, a forward distance to the remote position of the remote tissue portion.

The foregoing aspects of the disclosure are exemplary only and not intended to limit the scope of the disclosure.

### Additional Considerations

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A predictive needle insertion device comprising:
a needle having a proximal needle end and a distal needle end;
a probe movably coupled to the needle, the probe movable to a position extending beyond the distal needle end;
an actuator operable to actuate the probe to apply a mechanical force to a tissue composition, wherein the tissue composition includes a local tissue portion and a remote tissue portion, the local tissue portion being at a local position to the distal needle end and the remote tissue portion being at a remote position to the distal needle end;
a force sensor associated with the probe, the force sensor configured to detect a mechanical response to the mechanical force, the mechanical response being indicative of a resistive force;
a position sensor associated with the probe, the position sensor configured to measure an insertion distance of the probe beyond the distal needle end;
a processor communicatively coupled to the force sensor and the position sensor, the processor configured to receive sensor data indicative of the mechanical response to the mechanical force and the insertion distance of the probe; and
a non-transitory program memory communicatively coupled to the processor and storing executable instructions that, when executed by the processor, cause the processor to predict, based on the sensor data, a forward distance to the remote position of the remote tissue portion.

2. The predictive needle insertion device of claim 1, wherein the remote tissue portion is bone.

3. The predictive needle insertion device of claim 1, wherein the processor, executing the instructions, predicts the forward distance to the remote position of the remote tissue portion without the distal needle end contacting the remote tissue portion.

4. The predictive needle insertion device of claim 1, wherein the probe is configured to apply the mechanical force directionally forward at the local position of the local tissue portion.

5. The predictive needle insertion device of claim 1, wherein the probe is configured to apply the mechanical force to the tissue composition directionally forward beyond the distal needle end.

6. The predictive needle insertion device of claim 1, wherein the mechanical force is one of a plurality of multi-frequency forces, and wherein the actuator is further operable to actuate the probe periodically to apply the plurality of multi-frequency forces during a corresponding plurality of actuation iterations.

7. The predictive needle insertion device of claim 6, wherein each of the plurality of actuation iterations includes the probe extending and retracting along an axis associated with the needle.

8. The predictive needle insertion device of claim 6, wherein a plurality of frequencies of the plurality of multi-frequency forces is determined via step-input actuation.

9. The predictive needle insertion device of claim 8, wherein the step-input actuation is based on a sinusoidal signal provided to the actuator.

10. The predictive needle insertion device of claim 6, wherein a plurality of frequencies of the plurality of multi-frequency forces is determined via varied sinusoidal frequencies provided to the actuator.

11. The predictive needle insertion device of claim 1, wherein the mechanical force is one of a plurality of forces applied to the tissue composition and the resistive force is a near-steady-state response received during a zero-frequency data collection actuation of the probe over long time scale observation.

12. The predictive needle insertion device of claim 1, wherein the probe is capable of retracting into the distal needle end.

13. The predictive needle insertion device of claim 1, wherein the probe is an inter-needle probe operable to extend through the proximal needle end and the distal needle end.

14. The predictive needle insertion device of claim 1, wherein the needle is a 17 gauge needle.

15. The predictive needle insertion device of claim 1, wherein the needle is a disposable needle and the probe is a disposable probe, wherein each of the disposable needle and the disposable probe are removably coupled to the predictive needle insertion device.

16. The predictive needle insertion device of claim 1, wherein the needle is operable to receive a catheter through the proximal needle end and the distal needle end.

17. The predictive needle insertion device of claim 1, further comprising a display.

18. The predictive needle insertion device of claim 17, wherein the display includes an indicator light.

19. The predictive needle insertion device of claim 17, wherein the display includes a display screen.

20. The predictive needle insertion device of claim 17, wherein the display provides an indication of the forward distance to the remote position of the remote tissue portion.

21. The predictive needle insertion device of claim 17, wherein the display provides an alert indicating that the distal needle end is within a threshold distance from the remote tissue portion.

22. The predictive needle insertion device of claim 1, wherein the processor is an external processor external to a casing of the predictive needle insertion device.

23. The predictive needle insertion device of claim 22, wherein the external processor receives the sensor data via wireless communication.
